(19) United States
(12) Patent Application Publication
(10) Pub. No.: US 2007/0259114 A1
(43) Pub. Date: Nov. 8, 2007

(54) Partially coated workpieces and method and system for making the same

(75) Inventors: Anthony Malone, Oranmore (IE); Dave McMorrow, Galway City (IE); Barry Heaney, Ballybrit (IE); Timothy O'Connor, Claregalway (IE); Aiden Flanagan, Kilcolgan (IE)

Correspondence Address: KENYON & KENYON LLP, 1500 K STREET N.W., SUITE 700, WASHINGTON, DC, 20005, US

(73) Assignee: BOSTON SCIENTIFIC SCIMED, INC.

(21) Appl. No.: 11/415,109

(22) Filed: May 2, 2006

(51) Int. Cl. A61L 33/00; B05D 3/12; B05D 3/00; B05D 3/10

(52) U.S. Cl. 427/271; 427/299; 427/240; 427/290; 427/307; 427/2.1; 427/2.24

(57) ABSTRACT

The present invention is directed to methods, processes, and systems for coating portions of a workpiece as well as to workpieces that have themselves been coated in accord with the invention. Under these methods and processes of the invention, a workpiece may be rotated to drive coating away from a non-target surface. In some embodiments, surfaces of the workpiece may be pre-treated. Still further, in accord with the embodiments of the present invention, the workpiece may be positioned in a treatment chamber. In still other embodiments, the workpiece may be an implantable medical device and the coating may include a therapeutic.

### TECHNICAL FIELD

The present invention generally relates to partially coated workpieces and methods and systems for partially coating a workpiece with a coating or other treatment. More specifically, the present invention relates to workpieces, such as implantable medical devices, and methods and systems for coating these medical devices, wherein a treatment or other coating is applied to some but not all surfaces of the workpiece during a coating process.

### BACKGROUND

Coating workpieces is an often repeated procedure in contemporary manufacturing. Workpieces may be coated by methods that include tumble coating, spray coating, dip coating, and electrostatic spraying. During each of these procedures coating is applied to the workpiece prior to the workpiece being used for an intended purpose.

When the workpiece is formed partially or completely out of lattice struts or some other open framework, each of the faces of these struts or framework is exposed to the coating and coated during the coating methods listed above. By exposing each face of the workpiece to the coating being applied, each exposed face will be covered during the coating process.

When the workpiece being coated is an implantable medical device, such as a stent, all faces of the struts that comprise the stent are coated when using the coating systems identified above. For example, when dip coating is used, each face of the stent struts will be exposed to the coating. This coating will remain when the stent is removed from the dip and will dry on each face of the struts. Coating may also remain in the spaces between the struts. This phenomenon is sometimes called “webbing.” Here, not only are the individual struts covered, but some or all of the spaces between the struts are spanned by the coating as well.

### BRIEF DESCRIPTION

The present invention is directed to methods, processes, and systems for coating portions of a workpiece as well as to workpieces that have themselves been coated in accord with the invention. In accord with the invention, for example, some or all outer surfaces of a workpiece, such as a medical implant, may be coated with a therapeutic while inner surfaces of the implant, which are not targeted for coating, may not be coated.

Under methods and processes of the invention, a workpiece may be rotated as it is being coated to drive coating away from non-target surfaces of the workpiece. In other words, as a workpiece, such as a stent is coated, it may be spun such that surfaces of the stent that initially receive coating while the coating is applied, may not longer be coated once the coating is dried because the coating is removed from non-target areas of the stent by forces created from the rotation of the stent. In some embodiments, surfaces of the workpiece may be pre-treated, may have different degrees of smoothness or may be both pre-treated and have different degrees of smoothness. Still further, the workpiece may also be positioned in a treatment chamber during portions or all of the treating and coating process. As noted, the workpiece may be an implantable medical device and the coating may include therapeutic, the workpiece may be other devices as well.

The invention may be embodied in numerous devices and through numerous methods and systems. The following detailed description, which, when taken in conjunction with the annexed drawings, discloses examples of the invention. Other embodiments, which incorporate some or all of the features as taught herein, are also possible.

### BRIEF DESCRIPTION OF THE DRAWINGS

Referring to the drawings, which form a part of this disclosure:

FIG. 1a shows a workpiece holder connected to a motor shaft that may be employed in accord with the present invention;

FIG. 1b shows a workpiece positioned on the holder of FIG. 1a in accord with embodiments of the present invention;

FIG. 2a is a cross-sectional view of a portion of a coated strut from a medical device that has been coated in accord with the present invention;

FIG. 2b is a cross-sectional view showing the coated strut of FIG. 2a after a second coating has been applied as may be employed in accord with the present invention;

FIG. 2c is a side-view of an arterial stent, which is a medical device that may be coated in accord with the present invention;

FIG. 3a shows an electroplating process that may be employed in accord with the present invention;

FIG. 3b is an end-view of a portion of a coated strut from a medical device that has been pre-treated in accord with the present invention;

FIG. 3c shows a workpiece being sandblasted in accord with the present invention;

FIG. 3d is another end-view of a portion of a coated strut from a medical device that has been pre-treated in accord with the present invention;

FIG. 3e shows a workpiece being spray coated with a polymer adhesion promoter in accord with the present invention;

FIG. 3f is still another end-view of a portion of a coated strut from a medical device that has been pre-treated in accord with the present invention;

FIG. 4a shows a spray coating nozzle and motor as may be employed to coat a workpiece in accord with the invention;

FIG. 4b shows a dispensing nozzle and motor as may be employed to coat a workpiece in accord with the present invention;

FIG. 5 shows a dip coating system that may be employed to coat a workpiece in accord with the present invention;

FIG. 6a is a side-view in partial cross-section of a motor and a workpiece positioned in a treatment chamber as may be employed to coat the workpiece in accord with the present invention;

FIG. 6b is a side-view, in partial cross-section, of the motor and treatment chamber of FIG. 6a showing a workpiece holder positioned inside the treatment chamber; and

FIG. 7 is a side-view, in partial cross-section, of a workpiece immersed in a non-compressible fluid, a motor, a treatment chamber, and a dispensing member which dispenses coating, as may be employed in accord with the present invention.

### DETAILED DESCRIPTION

The present invention regards coating one or more surfaces of a workpiece while not coating other surfaces of the workpiece. In some embodiments this may include coating the outside or side surfaces of the workpiece while not coating the inside surfaces of the workpiece. By coating in this fashion the amount of coating resident on the workpiece may be reduced in some cases. This can be useful when the amount of coating resident on the workpiece is metered or is otherwise of interest. For example, if the workpiece is a stent and the coating contains therapeutic a reduction in coating may allow the therapeutic to be delivered in a more targeted fashion after the stent is implanted at a target site. The limited use of coating can also conserve coating materials, which themselves may be valuable.

The selective coating of a workpiece may be accomplished in various ways in accord with the present invention. For example, the workpiece may be rotated before the coating dries or otherwise cures. This rotation may act to drive coating away from a non-target surface. The workpiece may be pretreated in accord with the present invention as well. This pretreatment may act to repel or prevent coating from adhering to one or more surfaces of the workpiece. The workpiece may also be pretreated to facilitate the adhesion or attraction of coating to one or more surfaces of the workpiece. Pretreating may include various steps such as polishing, roughening, and applying polymer adhesion promoters.

The workpiece may be positioned in a treatment chamber when practicing the present invention and both compressible and non-compressible fluids may be supplied to the treatment chamber to improve coating distribution on targeted surfaces. In addition to coating target areas, the invention may also be used to retard “webbing” between areas that are coated. There are numerous other benefits of and uses for the present invention.

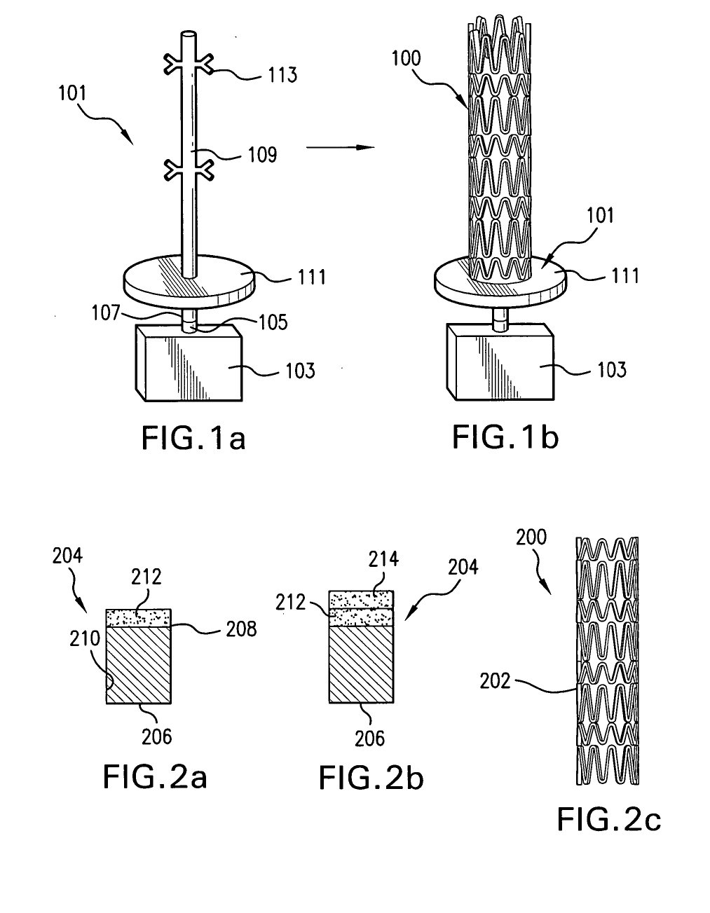

FIG. 1a is a side-view of a workpiece holder 101 in accord with the present invention. Evident in FIG. 1a are a motor 103, a motor shaft 105, a first shaft 107, and a second shaft 109. Also evident in FIG. la are arms 113, and cylindrical platform 111. As can be seen, the shaft 107 in this figure extends downwardly in a direction perpendicular to the platform 111 and the first shaft 107 connects with the motor shaft 105. The connection between the first shaft 111 and motor shaft 105 can be any of a variety of connections including flanges and fasteners. As can also be seen, the second shaft 109 in this figure extends upwardly in a direction perpendicular to the platform 111.

In FIG. 1a, the second shaft 109 has arms 113 extending outwardly and horizontally. The arms 113 in FIG. 1a include substantially V-shaped end portions for contacting a surface of a workpiece. These V-shaped portions are an example of the supports that may be used as other shapes, sizes, and configurations of these portions as well as the shafts 107, 109 and arms 113 can be also used in accord with the invention.

In other embodiments, which are not shown, the second shaft 109 may contact surfaces of the workpiece in a variety of other ways. For example, the second shaft 109 may not have arms and can be expandable and compressible to fit inside a workpiece. Thus, in a collapsed position, the workpiece may be placed on the shaft and removed and in an expanded position the workpiece may be supported during the coating process. The components of the holder 101 can also be fabricated from various materials including polymeric and metallic materials. Likewise, the components can be any suitable size and/or shape.

The motor 103 may be any machine that converts energy into mechanical energy to impart motion. In this instance, the motor 103 converts electrical energy into mechanical energy to impart rotary motion to the workpiece. In still other examples, which are not shown, the motor 103 may use mechanical energy (e.g., a crank) to impart rotation to the holder 101. The motor shaft 105 may be rotatable clockwise and/or counterclockwise.

FIG. 1b shows the workpiece holder 101 of FIG. 1a with a workpiece 100. Here the workpiece is an arterial stent. As seen in FIG. 1b, the workpiece 100 may be positioned on the platform 111 and over the arms 113 of the holder 101. In this instance, the workpiece 100 is positioned on the holder 101 and a first surface of the workpiece 100 contacts the arms 113. Although not shown, an additional securing element may also be provided to prevent movement of the workpiece 100 when being rotated. While the workpiece 100 is orientated vertically other orientations are possible when practicing the invention.

FIG. 2a is a side sectional view of a strut of a stent that may be coated in accord with the present invention. The strut 204 in FIG. 2a has an inner surface 206, an outer surface 208, and two cut faces 210. Also shown on the strut 204 is a coating 212. As can be seen, the coating 212, covers only one face of the strut 204.

FIG. 2b shows another example of how a coating may be applied in accord with the invention. In FIG. 2b, a first coating 212 and a second coating 214 have been applied to the strut 204. As can be seen, the first coating 212 is in contact with the strut 204 while the second coating 214 is in contact with the first coating 212 and further covers the outer surface 208 of the strut 204. This second coating 214 may be applied in accord with the processes and methods of the present invention. It may also be applied with different methods and processes. In this example, as well as with the others described herein, if a second coating 214 is employed this coating may comprise the same materials as the first coating 212 and it may differ from the materials used for the first coating 212. In still other examples the coating may be applied in other patterns as well. For example, it maybe applied to opposing cut faces 210 and not the outer surface 208, likewise it may be applied to both cut faces 210 and the outer surface 208. In a exemplary embodiment, the outer surface 208 is coated and the two cut faces 210 as well as the inner surface 206 are not.

FIG. 2c is a side view of an implantable aortic stent including a lattice portion 202 that may be coated in accord with the invention. The stent may be porous or have portions thereof that are porous. The struts 204 shown in FIGS. 2a and 2b are struts that may comprise and make up this stent. The stent may be self-expanding, mechanically expandable, or a hybrid stent which may have both self-expanding and mechanically expandable characteristics. The stent may be made in a wide variety of designs and configurations, and may be made from a variety of materials including plastics and metals.

Various methods may be employed for delivery and implantation of the stent. For instance, a self-expanding stent may be positioned at the distal end of a catheter around a core lumen.

Self-expanding stents may be typically held in an unexpanded state during delivery using a variety of methods including sheaths or sleeves which cover all or a portion of the stent. When the stent is in its desired location of the targeted vessel the sheath or sleeve is retracted to expose the stent which then self-expands upon retraction.

Another method includes mounting a mechanically expandable stent on an expandable member, such as a dilatation balloon provided on the distal end of an intravascular catheter, advancing the catheter through a patient's vasculature to the desired location within the patient's body lumen, and inflating the balloon on the catheter to expand the stent into a permanent expanded condition.

One method of inflating the balloon includes the use of inflation fluid. The expandable member is then deflated and the catheter removed from the body lumen, leaving the stent in the vessel to hold the vessel open.

While the workpiece 200 shown in these initial figures is a stent, many other workpieces 200 may be coated in accord with the invention. For example, other medical devices that may be coated include filters (e.g., vena cava filters), stent grafts, vascular grafts, intraluminal paving systems, implants and other devices used in connection with drug-loaded polymer coatings. Likewise, the workpeice 200 may not be an implantable medical device but may, instead, be another piece that needs to be coated only on certain pre-selected surfaces. In some instances these medical devices or other workpieces 200 may be made from conductive materials and in other instances they may not be. For example, they may be made from polymers or ceramics.

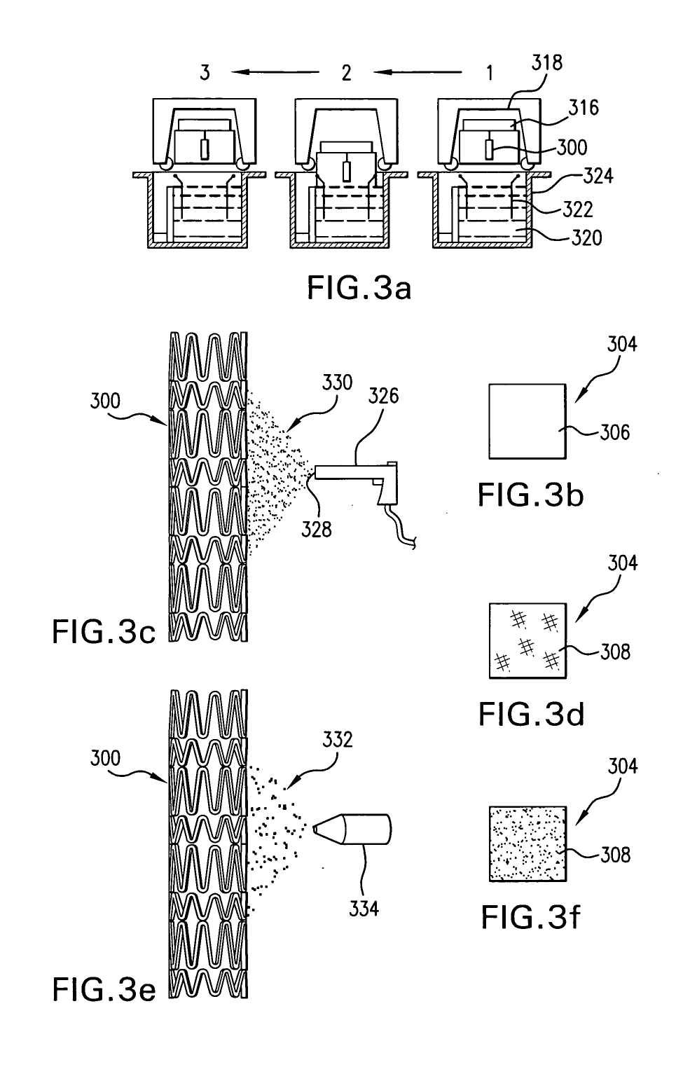

FIG. 3a is a side-view illustrating the workpiece during a pre-treatment step that may be employed in accord with the present invention. As seen in FIG. 3a, the workpiece 300, such as an arterial stent, may be subjected to a pre-treatment process. The workpiece 300 may be pre-treated by immersing the workpiece 300 in a bath 320 of a tank 324. Accordingly, the workpiece 300 may be removably mounted to a processing fixture 316 within a transfer carriage 318. The processing fixture 316 may be moveable within an enclosure of the transfer carriage 112. To begin a pre-treatment process such as electropolishing, the transfer carriage 318 may be moved over a bath 320 into position (1). In the instant case, the bath 320 is an electropolishing bath that houses electrodes 322, however, any suitable bath can be used depending upon the intended use of the workpiece 300. The processing fixture 316 may then lower the workpiece 300 and the enclosure into the bath 320, as shown in position (2). The enclosure may be lowered until it is just above the level of the electropolishing bath 320. A current may then be applied to the bath 320 so that the workpiece 300, which is submerged, is electropolished. The workpiece 300 may then be removed from the bath 320 as shown in position (3).

The electropolishing pre-treatment process of FIG. 3a polishes the surface of the workpiece. The polishing of a target surface of the workpiece 300 may reduce the “wettability” of the surface. In other words, it may be more difficult to coat a surface with reduced “wettability.”

As seen in FIG. 3b, following electropolishing, a surface of a strut 304 is generally smooth. In the example the inner surface 306 of the strut 304 is smooth. Therefore, coating may be repelled from the inner surface 306.

The pre-treatment step may also be applied with different methods and processes. For example, the workpiece 300 can be roughened during pre-treatment. Roughening the workpiece 300 may increase the “wettability” of the target surface of the workpiece 300. Therefore, the roughened surface may facilitate the coating of a target surface.

As seen in FIG. 3c, the workpiece 300 can be roughened by sandblasting. Here, a sandblasting gun 326 including a nozzle 328 may be used to roughen a surface of the workpiece 300. In FIG. 3c. the nozzle 328 may be directed towards an outer surface 308 of the workpiece 300 to direct sand 330 against the outer surface 308 to roughen the surface 308. The sand 330 roughens the outer surface 308 of the strut 304.

As described herein, the outer surface 308 may be roughened to increase “wettability.” Therefore, coating of the outer surface 308 may be facilitated. In still other examples, not shown, the workpiece 300 may be roughened with various conventional surface deposition techniques including etching and electroplating.

FIG. 3e shows an example of the workpiece 300 being sprayed with a polymer adhesion promoter 332. A nozzle 334 may be used to direct the polymer adhesion promoter 332 towards the workpiece 300.

FIG. 3f is an end-view showing an outer surface 308 of a strut 304 following the polymer adhesion promoter 332 application. The polymer adhesion promoter 332 also may facilitate the coating of a target surface of the workpiece. In other examples, which are not shown, polymer adhesion promoters 332 may be applied to the workpiece using other applications including etching or electroplating.

The invention may be pre-treated using any of numerous processes and methods. For example, the workpiece 300 may be pre-treated by mechanical abrading and chemical etching processes.

Mechanical abrading may be performed using any abrasive product or material which can either remove a layer, polish, or roughen a surface of a workpiece 300. The abrading process may be done by hand. For example, a non-rotary block or pad may be used. Alternatively, the abrading process may be performed with the assistance of a machine. For instance, a tool using an endless band of abrasive material or a rotary cylinder or disk may be used.

Chemical etching may also be used. Chemical etching involves the use of a chemical etchant to remove a layer, polish, or roughen a surface of a workpiece 300. For example, the workpiece 300 may be immersed in a bath of chemical etchant to polish the workpiece 300. Any etchant may be used including isotropic or anisotropic etchants. The workpiece 300 may also be contacted with ions from a plasma (e.g., nitrogen, chlorine, or boron trichloride) or chemically milled.

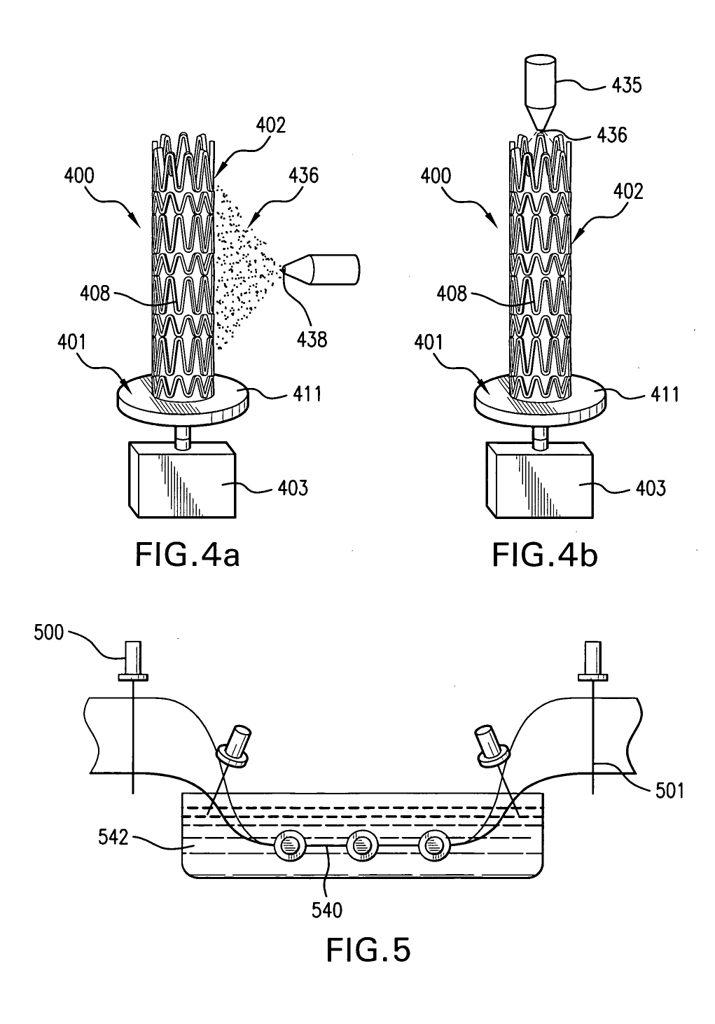

FIG. 4a shows another step that may be employed when practicing the invention. This step includes applying a coating 436 to a target surface of the lattice portion 402 of the workpiece 400. In this example, the surface is the outer surface 408 of the lattice portion 402. The coating of the outer surface 408 can be applied to the lattice portion 402 by various methods including, but not limited to, dipping, spraying, rolling, brushing, electrostatic plating or spinning, vapor deposition, air spraying including atomized spray coating, and spray coating using an ultrasonic nozzle.

In the example of FIG. 4a, a spray coating application is utilized. In this instance, the spray coating 436 is applied while the workpiece 400 is rotating, however, the coating can be applied prior to rotation. Spraying parameters such as atomization pressure and the distance between the nozzle 438 and workpiece 400 can be adjusted to vary the thickness of the coating 436. While the coating 436 is being applied, the motor 403 can be used to rotate the workpiece 400 in a clockwise and/or counterclockwise direction to drive coating 436 away from the inner surface of the workpiece 400.

In the example of FIG. 4b, a dispensing member 435 is used to apply the coating. In this instance, the coating 436 may be applied statically or dynamically. In other words, the coating can be applied before or while the workpiece 400 is rotating. The motor 403 in this case can also be used to rotate the workpiece 400 in a clockwise and/or counterclockwise directions to drive coating 436 away from the inner surface of the workpiece 400. The dispensing member 435 can be any suitable injection device wherein suitable examples include needles and syringes.

As FIGS. 4a and 4b illustrate, the coating can be selectively applied during rotation or prior to rotation. As the workpiece 400 is rotated, centrifugal forces experienced by the coating 436 drive the coating 436 towards the outer surface 408 and cut faces of the lattice portion 402. To improve performance or to achieve desired results, various process parameters can be controlled. For example, coating solution characteristics, spin speed, and spin time can be varied to improve coating of targeted surfaces.

One parameter that may be varied are the coating solution characteristics. For example, the coating solution viscosity may be important in determining how the coating spreads and deposits on the outer surface 408 and cut faces of the lattice portion 402. In some instances, viscosity can be controlled by varying the elements of the coating 436. For example, some coating 436 includes organic solvents into which a therapeutic may be dissolved. The organic solvent can be varied to control viscosity. In still other instances, the percentage of solids, the addition of biocompatible surfactants, and the release of the therapeutic can vary to control viscosity.

Another parameter that can change is the density of the coating 436. For example, if a coating 436 including therapeutic and polymer were used, the therapeutic may be suspended in the polymer. Therefore, when the denser therapeutic experiences centrifugal force from being rotated, the therapeutic may be forced to the outer surface 408 of the workpiece 400. This results in concentrated therapeutic on the outer surface of the workpiece 400. Therefore, the amount of therapeutic utilized can be minimized.

Still another parameter that may be varied is the speed or revolutions per minute (RPM) of the motor shaft and/or holder. The RPM can be varied to affect the degree of radial centrifugal force applied to the coating 436. Additionally, the velocity and turbulence of the air which surrounds the workpiece 400 can also be controlled. In FIGS. 4a and 4b, the RPM range may preferably be between about 30 and 3,000 RPMs.

Yet still another parameter that can be varied is the duration of time the workpiece 400 is rotated. Spinning duration can affect the thickness and positioning of the coating 436 on the outer surface 408 of the lattice portion 402. For example, in some instances, the spin time may preferably be around five minutes.

FIG. 5 illustrates steps wherein coating may be applied to a plurality of workpieces 500 by advancing an endless belt 540 through a coating bath 542. As a result, each workpiece 500 may be dip coated. Following dip coating, each workpiece 500 and holder 501 may be rotated as described herein. These steps may be advantageous in processes where multiple workpieces 500 are being manufactured. Additionally, in accord with the invention, a solid porous coating including therapeutic may also be applied. When the workpiece 500 is removed from the coating bath 542 and rotated, the therapeutic can then migrate to the outer surface porous layers while still remaining within the porous structures. This method may be advantageous in varying the depth of coating therapeutic.

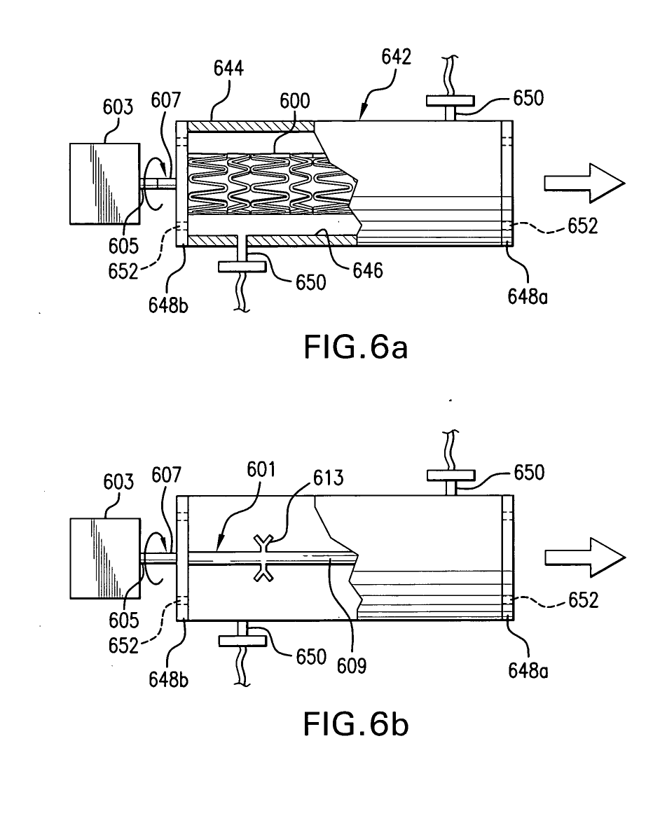

FIG. 6a is a side view of a horizontally orientated treatment chamber 642 in accord with an embodiment of the present invention. In this embodiment, the treatment chamber 642 includes an outer wall 644, an inner wall 646, and end plates 648a, 648b. The outer wall 644 may include a fluid passage or passages 650 which may be designed and sized to allow compressible fluids, such as air, nitrogen, carbon dioxide, and other compressible gases, to pass from outside the outer wall 644 to inside the inner wall 646 and into the treatment chamber 642.

The end plates 648a, 648b may include exhaust ports 652, which may be sized and designed to allow compressible fluid entering the treatment chamber to be exhausted from the chamber. At least one of the end plates 648a, 648b, in the instant case 648a, may be rotatably coupled to the treatment chamber 642 so that it may swing away from the treatment chamber to allow a workpiece 600 to be positioned within the treatment chamber. The arrow in FIG. 6a. indicates the direction in which the end plates move.

As seen in FIG. 6a, the other end plate 648a, 648b (here 648b), may be configured to rotatably connect to the motor shaft 605. A first shaft 607 extends from the end plate 648b to integrally connect with the motor shaft 607. As a result, the entire treatment chamber 642 may be rotated.

As seen in FIG. 6b, a second shaft 609 extends from end plate 648b to form a workpiece holder 601. The holder 601 includes arms 613 to support the workpiece 600. A variety of arrangements may be envisioned to support the workpiece 600 within the treatment chamber 642 in accord with the embodiments. For example, the workpiece 600 can be levitated by the fluid supplied to the treatment chamber 642.

As indicated above, when one of the end plates 648a is in an open position, a workpiece 600 may be positioned into the treatment chamber 642 and onto the holder 601. Once the workpiece 600 has been placed within the treatment chamber 642, it may then be treated, coated or otherwise interfaced with a therapeutic or other material. In the instant case, the workpiece 600 can be coated prior to or while positioned inside the treatment chamber 642. During or after the workpiece 600 has been interfaced with the coating, compressible fluid may be supplied to and exhausted from the chamber to facilitate drying and/or evaporation of the coating.

Once the workpiece 600 has been treated and removed, another workpiece 600 may be positioned inside the chamber for treatment. This treatment cycle may be repeated as necessary. Alternatively, the treatment chamber 642 may be designed or constructed to be used only a single time and then discarded.

As in the above example, the coating may be injected through the same fluid passages 650 that are injecting the compressible fluid into the treatment chamber. Thus, the fluid passages may be carrying therapeutic, compressible fluids coatings or a combination. Where both therapeutic, coatings or both and compressible fluids are being carried through the same fluid passage the therapeutic or coatings may be mixed with the compressible fluid upstream of the fluid passage 650 or may be atomized at or near the entrance or exit of the fluid passage 650.

In still other embodiments, therapeutic may also be injected via fluid passages that do not contain or are not carrying compressible fluid.

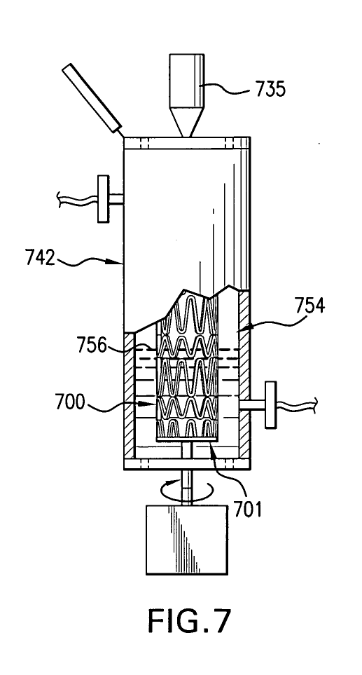

As shown in FIG. 7, a dispensing member 735 may be used to dispense coating to an inner portion of the workpiece 700. The coating may be dispensed while the workpiece 700 is rotating (e.g., dynamically) or while the workpiece 700 is stationary (e.g., statically). Static or dynamic dispensing may depend on the characteristics of the coating and the thickness of the coating required. The dispensing member 735 may be any suitable component including injection members and syringes. In the instant case, the workpiece 700 is stationary.

In this example, non-compressible fluid 754 is also supplied to the treatment chamber 742. When the workpiece 700 is rotated, the fluid moves toward the outer wall 756 of the treatment chamber 742 to form a boundary around a surface of the workpiece 700. Therefore, the concentration of the coating on a target surface may improve. In FIG. 7, the motor shaft 705 extends through the treatment chamber 742 and rotates the workpiece holder 701, however, other arrangements described herein are plausible.

The term “treatment chamber” as used herein may be any vessel having defined walls with inside surfaces. A treatment chamber may be made from various materials including clear, translucent, and opaque polymers, metals, and ceramics. Clear polymers, which provide for the internal viewing of implants being coated or impregnated with therapeutics in the treatment chamber, may be used in an exemplary embodiment.

The treatment chamber may be preferably cylindrical but it may be other shapes as well. These shapes may include octagons, other multi-sided polygons, ovals, and non-symmetrical shapes. Furthermore, the treatment chamber may be sized to hold one or more implants.

In an exemplary embodiment, a treatment chamber may be sized to allow implants to be positioned end to end next to one another but not side by side. In other words, in an exemplary embodiment where both the implants and the treatment chamber are cylindrical, the inside diameter of the treatment chamber may be slightly larger than the outside diameter of the implant to be coated. The flow rate and pressure of the compressible fluid injected into the treatment chamber and the size and placement of the fluid passages may be adjusted to accommodate the size, shape, and weight of the implant to be coated. It may also be adjusted depending upon the compressible fluid being used and the pressure developed within the coating chamber. The size and placement of the exhaust ports may also affect the flow rate and pressure of the compressible fluid being used. Still further, the implants may be loaded into the chamber in various orientations, e.g., forward, backward, open, and closed (in the case of an expandable implant).

While various embodiments have been described, other embodiments are plausible. It should be understood that the foregoing descriptions of various examples of the rotating member and treatment chamber are not intended to be limiting, and any number of modifications, combinations, and alternatives of the examples may be employed to facilitate the effectiveness of the coating of target surfaces of the workpiece.

The coating, in accord with the embodiments of the present invention, may comprise a polymeric and or therapeutic agent formed, for example, by admixing a drug agent with a liquid polymer, in the absence of a solvent, to form a liquid polymer/drug agent mixture. A suitable list of drugs and/or polymer combinations is listed below. The term “therapeutic agent” as used herein includes one or more “therapeutic agents” or “drugs.” The terms “therapeutic agents” or “drugs ” can be used interchangeably herein and include pharmaceutically active compounds, nucleic acids with and without carrier vectors such as lipids, compacting agents (such as histones), viruses (such as adenovirus, andenoassociated virus, retrovirus, lentivirus and α-virus), polymers, hyaluronic acid, proteins, cells and the like, with or without targeting sequences.

Specific examples of therapeutic agents used in conjunction with the present invention include, for example, pharmaceutically active compounds, proteins, cells, oligonucleotides, ribozymes, anti-sense oligonucleotides, DNA compacting agents, gene/vector systems (i.e., any vehicle that allows for the uptake and expression of nucleic acids), nucleic acids (including, for example, recombinant nucleic acids; naked DNA, cDNA, RNA; genomic DNA, cDNA or RNA in a non-infectious vector or in a viral vector and which further may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic and anionic polymers and neutral polymers that are selected from a number of types depending on the desired application. Non-limiting examples of virus vectors or vectors derived from viral sources include adenoviral vectors, herpes simplex vectors, papilloma vectors, adeno-associated vectors, retroviral vectors, and the like. Non-limiting examples of biologically active solutes include anti-thrombogenic agents such as heparin, heparin derivatives, urokinase, and PPACK (dextrophenylalanine proline arginine chloromethylketone); antioxidants such as probucol and retinoic acid; angiogenic and anti-angiogenic agents and factors; anti-proliferative agents such as enoxaprin, angiopeptin, rapamycin, angiopeptin, monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, acetyl salicylic acid, and mesalamine; calcium entry blockers such as verapamil, diltiazem and nifedipine; antineoplastic/antiproliferative/anti-mitotic agents such as paclitaxel, 5-fluorouracil, methotrexate, doxorubicin, daunorubicin, cyclosporine, cisplatin, vinblastine, vincristine, epothilones, endostatin, angiostatin and thymidine kinase inhibitors; antimicrobials such as triclosan, cephalosporins, aminoglycosides, and nitrofurantoin; anesthetic agents such as lidocaine, bupivacaine, and ropivacaine; nitric oxide (NO) donors such as linsidomine, molsidomine, L-arginine, NO-protein adducts, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warfarin sodium, Dicumarol, aspirin, prostaglandin inhibitors, platelet inhibitors and tick antiplatelet factors; vascular cell growth promotors such as growth factors, growth factor receptor antagonists, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; survival genes which protect against cell death, such as anti-apoptotic Bcl-2 family factors and Akt kinase; and combinations thereof. Cells can be of human origin (autologous or allogenic) or from an animal source (xenogeneic), genetically engineered if desired to deliver proteins of interest at the insertion site. Any modifications are routinely made by one skilled in the art.

Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides can also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that can be injected, or whose DNA can be incorporated, include without limitation, angiogenic factors and other molecules competent to induce angiogenesis, including acidic and basic fibroblast growth factors, vascular endothelial growth factor, hif-1, epidermal growth factor, transforming growth factor ∀ and ∃, platelet-derived endothelial growth factor, platelet-derived growth factor, tumor necrosis factor ∀, hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for treating malignancies; and combinations thereof. Still other useful factors, which can be provided as polypeptides or as DNA encoding these polypeptides, include monocyte chemoattractant protein (“MCP-1”), and the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2,BMP-3,BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

As stated above, coatings used with the exemplary embodiments of the present invention may comprise a polymeric material/drug agent matrix formed, for example, by admixing a drug agent with a liquid polymer, in the absence of a solvent, to form a liquid polymer/drug agent mixture. Curing of the mixture typically occurs in-situ. To facilitate curing, a cross-linking or curing agent may be added to the mixture prior to application thereof. Addition of the cross-linking or curing agent to the polymer/drug agent liquid mixture must not occur too far in advance of the application of the mixture in order to avoid over-curing of the mixture prior to application thereof. Curing may also occur in-situ by exposing the polymer/drug agent mixture, after application to the luminal surface, to radiation such as ultraviolet radiation or laser light, heat, or by contact with metabolic fluids such as water at the site where the mixture has been applied to the luminal surface. In coating systems employed in conjunction with the present invention, the polymeric material may be either bioabsorbable or biostable. Any of the polymers described herein that may be formulated as a liquid may be used to form the polymer/drug agent mixture.

The polymer used in the exemplary embodiments of the present invention is preferably capable of absorbing a substantial amount of drug solution. When applied as a coating on a medical device in accordance with the present invention, the dry polymer is typically on the order of from about 1 to about 50 microns thick. In the case of a balloon catheter, the thickness is preferably about 1 to 10 microns thick, and more preferably about 2 to 5 microns. Very thin polymer coatings, e.g., of about 0.2-0.3 microns and much thicker coatings, e.g., more than 10 microns, are also possible. It is also within the scope of the present invention to apply multiple layers of polymer coating onto a medical device. Such multiple layers are of the same or different polymer materials.

The polymer of the present invention may be hydrophilic or hydrophobic, and may be selected from the group consisting of polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers.

Coatings from polymer dispersions such as polyurethane dispersions (BAYHDROL®, etc.) and acrylic latex dispersions are also within the scope of the present invention. The polymer may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment of the invention, the preferred polymer is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyisocyanates such that the devices become instantly lubricious when exposed to body fluids. In another preferred embodiment of the invention, the polymer is a copolymer of polylactic acid and polycaprolactone.

The examples described herein are merely illustrative, as numerous other embodiments may be implemented without departing from the spirit and scope of the exemplary embodiments of the present invention. Moreover, while certain features of the invention may be shown on only certain embodiments or configurations, these features may be exchanged, added, and removed from and between the various embodiments or configurations while remaining within the scope of the invention. Likewise, methods described and disclosed may also be performed in various sequences, with some or all of the disclosed steps being performed in a different order than described while still remaining within the spirit and scope of the present invention.

What is claimed is:

1. A method of coating preselected portions of a workpiece comprising:
identifying a first surface of the workpiece and a second surface of the workpiece;
exposing the first surface of the workpiece and the second surface of the workpiece to a coating; and
removing coating from a target area of the second surface of the workpiece but not from a target area of the first surface of the workpiece by rotating the workpiece.

2. The method of claim 1, wherein the second surface is pre-treated prior to rotating the workpiece to resist coating thereof and wherein the workpiece is expandable from a first configuration to a second configuration.

3. The method of claim 1, wherein the target area of the first surface is pre-treated to promote adherance of the coating to the first surface.

4. The method of claim 1, further comprising:
placing the workpiece on a workpiece holder prior to coating the first surface.

5. The method of claim 4, further comprising:
positioning the workpiece in a treatment chamber.

6. The method of claim 5 wherein the treatment chamber contains a non-compressible fluid when the workpiece is rotated.

7. The method of claim 1 wherein the workpiece contains a lattice having a plurality of struts, the first surface being an exposed outer surface of a lattice strut and the second surface being a cut face of the lattice strut.

8. The method of claim 2 wherein the pre-treatment includes mechanically abrading the second surface.

9. The method of claim 2 wherein the pre-treatment includes chemically etching the second surface.

10. A method of coating portions of a medical device comprising:
identifying a first surface of the medical device and a second surface of the medical device;
pre-treating the first surface to promote adherance of coating;
pre-treating the second surface to resist coating;
coating the first surface of the medical device with a coating;
coating the second surface of the medical device with a coating; and
rotating the medical device to remove coating from a target area of the second surface and to urge the coating towards a target area of the first surface.

11. The method of claim 10, further comprising:
placing the medical device on a medical device holder prior to coating the first surface, the medical device holder having at least one arm and a platform.

12. The method of claim 11, further comprising:
positioning the medical device in a treatment chamber.

13. The method of claim 12 wherein the treatment chamber contains a non-compressible fluid when the medical device is rotated.

14. The method of claim 12 wherein the treatment chamber contains a compressible fluid when the medical device is rotated.

15. A method of coating portions of a stent including a lattice, comprising:
identifying a first surface of the lattice and a second surface of the lattice;
pre-treating one of the first or second surfaces;
coating the lattice, including the pre-treated surfaces; and
rotating the stent to remove coating from a target area of the second surface of the lattice and to urge coating from the second surface towards a target area of the first surface of the lattice.

16. The method of claim 15 wherein the coating contains a therapeutic.

17. The method of claim 15 wherein pre-treating one of the first or second surfaces includes electro-polishing a surface.

18. The method of claim 15 wherein the stent is rotated in a treatment chamber and the treatment chamber contains a non-compressible fluid when the stent is rotated.

19. The method of claim 17 wherein the treatment chamber contains a compressible fluid when the stent is rotated.

20. The method of claim 15 further comprising: timing the rotation of the stent to control the thickness of the coating on the second surface.

21. The method of claim 15 wherein the stent is rotated while the stent is coated.
